(19) United States
(12) Patent Application Publication
(10) Pub. No.: US 2015/0182553 A1
(43) Pub. Date: Jul. 2, 2015

(54) USE OF POLYMER D-LACTIC ACID (PDLA) TO TREAT MALARIA

(71) Applicant: Joel Steven Goldberg, Hillsborough, NC (US)

(72) Inventor: Joel Steven Goldberg, Hillsborough, NC (US)

(21) Appl. No.: 14/304,205

(22) Filed: Jun. 13, 2014

Related U.S. Application Data
(60) Provisional application No. 61/922,393, filed on Dec. 31, 2013.

(51) Int. Cl. A61K 31/765

(52) U.S. Cl. CPC A61K 31/765

(57) ABSTRACT

The reaction of L-lactate and polymer D-lactic acid (PDLA) spontaneously and rapidly forms a stereocomplex. This versatile chemical reaction can disrupt glycolysis, the predominate form of ATP production in many disease states, and may have many uses including the treatment of malaria. PDLA can sequester lactate in the vicinity of hypermetabolic activity such as that associated with Plasmodium replication and phagocytosis. PDLA can inhibit Plasmodium activity or decrease Plasmodium survival. The reaction mechanism of L-lactate with PDLA may not be unique and other chiral polymers may sequester corresponding single unit enantiomers. If such reactions are found to occur, these chiral polymers may have the capacity to interrupt metabolic pathways as drugs. Although the stereocomplex reaction of PDLA with L-lactate will occur in all tissues with lactate, understanding the micro environments where hypermetabolic activity takes place makes it possible to modify administration, oligomer size and synthesize ester prodrugs of PDLA to more closely target areas of disease.

### CROSS-REFERENCES TO RELATED APPLICATIONS

This application claims the benefits of U.S. Provisional Patent Application No. 61/922,393 filed Dec. 3, 2013 each of which is incorporated herein by reference in its entirely.

### FEDERALLY FUNDED RESEARCH

Not applicable

### BACKGROUND OF THE INVENTION

Glycolysis is the primary source of glucose metabolism utilized by intra erythrocytic Plasmodium.^{1,2} The net products of glycolysis are ATP, hydrogen ions and pyruvate. Plasmodium, along with other cells, that need to sustain hypermetabolic states utilize glycolysis for energy production. Although glycolysis does not efficiently convert glucose to ATP on a molar basis, glycolysis provides a high flux of ATP. Formally considered only a waste product, L-lactate (lactate) in the presence of LDH1, found in Plasmodium, can be converted to pyruvate that may be an essential intermediate required to sustain hypermetabolic or hyperactive states. In previous inventions, selected oligomers of polymer D-lactic acid (PDLA) have been shown to form a stereocomplex with L-lactate in a spontaneous, non-enzymatic and rapid manner.³ In this invention, when lactate is sequestered by PDLA within the vicinity of Plasmodium, a stereocomplex is formed, glycolysis is interrupted, and the organism is impaired and may not survive.

In prior art, antimalarials have been developed to inhibit the virulence of malaria by targeting the formation of β hematin (dimer) and subsequent crystallization to hemozoin reactions.^{4,5} (Table 1) One proposed mechanism for the formation of hemozoin is through a non-enzymatic spontaneous chemical reaction that may be more difficult to target than a reaction that is dependent upon enzyme kinetics. The formation of the iron carboxylate bond of β hematin can be produced without an enzyme in vitro.⁶ A proposed mechanism at pH 4.5 is nucleophilic substitution of the carboxylic acid group of the heme propionic acid moiety with ferrous oxy heme (compound III) and water as a leaving group. (FIG. 1)

This proposed mechanism may be energetically favored.

| | |
|---|---|
| Σ bonds broken | — Σ bonds formed |

| | | | |
|---|---|---|---|
| Fe—O | 409 kJ/mol | H—O | 425 kJ/mol |
| C—O | 1073 kJ/mol | C—O | 1073 kJ/mol |
| | | H••• | 23.3 kJ/mol |

There are 2 F—O bonds broken and 2 H—O bonds and 4H . . . bonds formed per dimer. The average bond enthalpy for each F—O single bond from Fe═O is less than a single Fe—O bond. Therefore,

ΔH˜<818 kJ/mol−943 kJ/mol=−25 kJ/mol

The mechanism is consistent with the biomineralization concept of hemozoin formation previously proposed by Egan et al.⁶ This proposed non-enzymatic reaction could account for the partial response to treatment when hemozoin formation is blocked with present antimalarials since spontaneous non-enzymatic reactions may be more difficult to perturb than those that require an enzyme.

In prior art, antimalarials have been developed to interrupt folate metabolism. The malaria parasite has a high demand for nucleotides as precursors for DNA synthesis and is sensitive to antifolates that impair one carbon transfers. (Table 1)

In prior art, antimalarials have been developed to increase the availability of nitric oxide by administration of L-arginine. (Table 1)

In prior art, inhibitors of protein synthesis at the ribosome have been used to treat malaria. (Table 1)

TABLE 1
Some present antimalarials with proposed site of action

| | Hemozoin | Folate- | | Ribosome- | Nitric |
|---|---|---|---|---|---|
| | formation | DNA | Free radical | RNA | oxide |
| Quinine | + | | | | |
| Chloroquine | + | | | | |
| Pyrimethamine | | + | | | |
| Sulfonamides | | + | | | |
| Artemisinin | | | + | | |
| Doxyclycline | | | | + | |
| Clindamycin | | | | + | |
| L-arginine | | | | | + |

Disrupting glycolysis by sequestration of L-lactate with PDLA has some fundamental advantages over methods of prior art. The reaction of PDLA with L-lactate is spontaneous, non-enzymatic and rapid and reactions with similar characteristics in the field of medicinal chemistry are few. Plasmodium may have limited ability to adapt to such a spontaneous, non-enzymatic and rapid reaction that can interfere with the production of ATP.

### DESCRIPTION OF THE DRAWINGS

FIG. 1 shows a possible mechanism for the formation of hemozoin.⁷

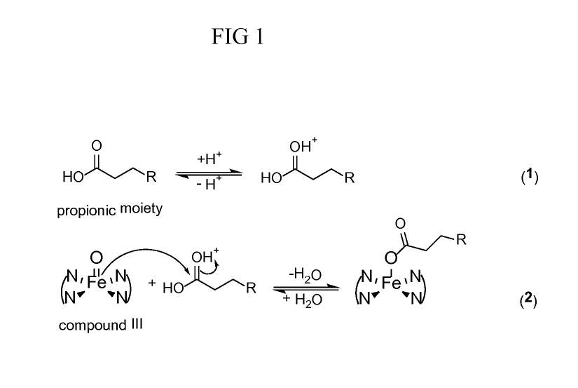

FIG. 2 shows the various pathways of pyruvate that is the precursor of lactate.⁸

### DETAILED DESCRIPTION OF THE INVENTION

Plasmodium infections, like other biological processes that require high sustained and burst energy, fill this requirement with ATP derived primarily from glycolysis. Examples of a diverse group of hyperactive biologic processes that like malaria require a need for energy production in this form include sprinting, neuronal excitation in mania and PTSD, firing of nociceptors in acute and chronic pain and multiplying cells in cancer, immune responses, bacterial and protozoan infections. As long as adequate glucose is present, glycolysis, often thought to be an inefficient system to generate ATP especially when compared to the Krebs cycle coupled to oxidative phosphorylation, can produce ATP at a rate sufficient to sustain these hyperactive states.

This concept can be more easily understood when one considers the energy requirements for a city. Similar to these hyperactive biological systems, a city requires high sustained power and burst or peak demand that cannot be completely predicted. These requirements need to be met for the society to function. Often peak demand is satisfied from expensive sources of electricity such as that produced by combustion of diesel fuel. Similar to distribution of electricity, Plasmodium infections and other hyperactivity states serve their energy needs by utilizing ATP that can be generated at a rapid rate with glycolysis even though the glucose cost to produce ATP is high.

Description of the Stereocomplex reaction of L-lactate with polymer-D-lactic acid (PDLA)

Previous art has shown that a stereocomplex is formed when oligomers of PDLA are combined with L-lactate and this reaction has been described as sequestering or “trapping” lactate. Trapping lactate with PDLA is an extraordinarily versatile chemical reaction that may have medical uses in the treatment of cancer and diseases of cerebral metabolism that require lactate as a fuel and as in this invention malaria. Non-medical uses may also apply. Whenever there is a system that utilizes lactate for production of ATP, sequestration of lactate will disrupt this system. Formation of the stereocomplex in some environments produces a crystalline like change in state. In this invention, lactate trapping disrupts glycolysis, the predominant method that Plasmodium utilizes for energy.

When negatively charged PDLA reacts with negatively charged L-lactate, the chiral force may overcome the repelling charge and hydrogen bond forces; thus a stereocomplex is formed. Furthermore it has been demonstrated that the PDLA dimer (n=2) will sequester lactate in an approximate ratio of 2 moles of PDLA to 1 mole of L-lactate and that the PDLA tetramer (n=4) does not sequester L-lactate. The formation of a stereocomplex between L-lactate and PDLA may not be a unique mechanism, rather other chiral polymers may complex single unit enantiomers. If this prediction is true, synthesizing chiral polymers may provide a method to sequester or trap levorotatory amino acids and/or dextrorotatory sugars. Since most organisms have evolved with L-enantiomers of amino acids and D-enantiomers of glucose, sequestering these unit enantiomers that are natural products of biochemical pathways may improve the pharmacologic treatment of many disease states. In fact, hetero-stereocomplexation has been demonstrated between L-configured polypeptides and PDLA.^{9,10} Chiral polymers could be future drugs.

Sequestration of lactate may disrupt many biological systems because lactate, once thought to be exclusively a waste product, is involved in many different functions:

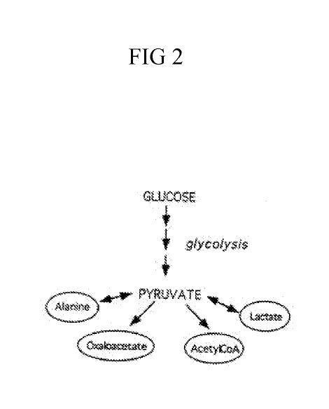

1. When PDLA sequesters L-lactate, the pathways that determine the fate of pyruvate are disrupted. (FIG. 2) The PDLA˜L-lactate stereocomplex does not provide lactate substrate feedback, and as a consequence, the system likely attempts to produce more lactate. In response to shifting the pyruvate to lactate equilibrium in favor of lactate, increased amounts of NAD+ and hydrogen ions will be produced. The hydrogen ions need to be buffered or effluxed and the NAD+ needs be reduced. The latter can occur by recycling the NAD+ to NADH as part of the reaction of glyceraldehyde 3-PO₄ to 1,3 biphosphoglycerate. The overall changes in ATP production from sequestration of lactate are difficult to predict since Acetyl-CoA can be produced from conversion of alanine

2. With sequestration lactate can no long serve as a fuel source. This could be important within hyperactive biological systems, such as Plasmodium replication, that expresses LDH1 (which converts lactate to pyruvate).

3. Upon sequestration, sequestered lactate can no longer function as a buffer to neutralize hydrogen ions that are waste products of glycolysis. In such systems as Plasmodium infection or tumor cell growth, efflux of hydrogen ions is required so that the cell can maintain electrical neutrality. If lactate cannot be used as a buffer the cell needs to find other alternatives probably through monocarboxylate transporters to accommodate the acid load.

4. Most likely, sequestered lactate cannot be shuttled. Lactate shuttling occurs in the central nervous system and sequestering lactate with PDLA may also have a role in the treatment of cerebral malaria and seizures.

5. Unlike lactate within the CNS, sequestered lactate cannot function as a neurotransmitter to bind to recently postulated presysnaptic lactate receptors or serve as a fuel for neuronal activity.¹¹

6. Sequestered lactate will interfere with gluconeogenesis.

The net outcome of sequestration of lactate is disruption of glycolysis and disruption of associated energy production.

Understanding the intricacies of energy transfer in response to sequestration of lactate in hyperactive systems such as glycolysis cannot be explained by classical thermodynamics because these systems are not in equilibrium. They are open systems where energy and matter are not isolated from the surroundings. Furthermore, the concentration of substrates, that can be both reactants and products, produce autocatalytic oscillation within the system. Non-equilibrium thermodynamics provides a better model of these systems but the complexities are such that models cannot be easily reduced to practice. However, inability to describe these systems with classical thermodynamics does not mean that informed predictions cannot be made when lactate is sequestered within such systems. This is especially true if one considers the microenvironment where reactions are to occur.

Microenvironments

Central Nervous System

Cerebral malaria is known be a major cause of morbidity and mortality. In the CNS, lactate shuttles from astrocytes to neurons. Whether this lactate serves as a neurotransmitter to increase neuronal firing or fuel for neurons the net result is to sustain a hyperexcitable state. PDLA can be targeted to the central nervous system by administration of oligomers of low molecular weight (<400 Daltons) that would fulfill some of the modified Lipinski criteria for drugs that cross the blood brain barrier (BBB). The PDLA may need to be modified and administered as an ester prodrug with conjugations to highly lipid cholesterol or conjugated to glucose to be actively transported. Such ester conjugations have been proven effective to transport GABA into the CNS.^{12,13} These differences in microenvironments between the CNS and the surroundings allows one to predict how a lipophilic derivative of PDLA, comprised of low molecular weight oligomers, may decrease lactate within the CNS in advance of performing the in vivo experiment.

Skin

Although the Plasmodium parasite is rarely associated with a skin lesion, the debilitated state of the host predisposes it to cutaneous lesions including skin ulcers and gum diseases. In this affliction, a mixture of multiple oligomers of topical PDLA can be effective at controlling the resulting pain presumably by sequestering lactate that functions as a fuel for sustained firing of action potentials. Topical application of PDLA as an analgesic has been demonstrated in the treatment of a wound of a human subject.

Intra Erythrocytic Malaria.

The intra erythrocytic form of malaria is not exposed to blood plasma and resides within the erythrocyte. The mechanism for PDLA to inhibit replication of Plasmodium may be three-fold: 1) Inhibit efflux of hydrogen ions from glycolysis by stereocomplexing lactate which serves as a buffer for this process 2) Direct interference of ATP production especially where LDH1 isoforms predominate. The latter is a complicated process in which the product of glycolysis, pyruvate, also is a reagent of Krebs cycle. Also NAD+, generated during the conversion of pyruvate to lactate is a reactant of the conversion of gluteraldehyle 3-PO₄ to 1,3 biphosphoglycerate. The rate equations of such autocatalytic systems are non-linear and the systems described by the reaction have an oscillatory behavior. Therefore the best one can predict is that the systems will be disrupted. 3) Inhibiting production of ATP. D-lactate (the product of PDLA hydrolysis) has been demonstrated to block mitochondrial respiration of L-lactate in the brain and heart and may also decrease ATP production available to Plasmodium.¹⁴

The pharmacokinetics of parenteral administered PDLA would include crossing, at minimum, membranes of three structures (endothelial: erythrocyte: Plasmodium) and the pH within each microsystem would be a major factor partitioning the drug. Partitioning the drug during the hyperactive liver stage of Plasmodium would require the drug to cross endothelium, Kupffer cell, hepatocyte and Plasmodium membranes.

Although sequestration of L-lactate by PDLA is a nonspecific reaction, understanding the microenvironment surrounding the hyperactive system, allows one to envision animal models and human administrations that may ultimately benefit the host. With known parameters of molecular weight, lipid solubility (log P or log D), pKa, hydrogen bond donor and acceptors and uptake in vessel rich and vessel poor tissue compartments, it may be possible to predictably target the use of PDLA within a non-equilibrium thermodynamic system.

To summarize, PDLA is able to sequester L-lactate in a nonspecific, non-enzymatic, spontaneous, and rapid way. Understanding the micro environment where such a reaction is needed along with traditional pharmacokinetic parameters, the pharmacodynamics of PDLA may be predicted. Although lactate has many functions, sequestration of lactate in malaria will disrupt glycolysis and inhibit Plasmodium replication. Interfering with glycolysis in non-hyperactive systems within the host is unlikely to cause significant side effects because these systems can survive with sustained low level ATP through oxidative phosphorylation if some pyruvate is produced when glycolysis is not completely inhibited. Also these systems can produce pyruvate from amino acids, such as alanine, relatively independent of glycolysis. Amino acids can then be converted to Acetyl-CoA and provide energy through oxidative phosphorylation. Because of poor lipid solubility, oligomers of PDLA are unlikely to penetrate the BBB, but could be modified as ester prodrugs conjugated to such moieties as cholesterol or glucose that may cross the BBB.

Toxicity of PDLA

The toxicity of PDLA in humans or animals has not been reported but the stereocomplex formed by PDLA and PLLA (PDLL or polylactide) is found in prosthetic devices. A metabolite of PDLA, D-lactic acid at average plasma concentrations of 7.98 mM, may be related to reversible symptoms of altered mental status, dysarthria, and ataxia.

Benefits to Society

Organisms need to sustain hyperactive or hypermetabolic systems in order to survive. The prime examples of which are the fight or flight states. However, when the hyperactive system becomes chronic it may often be deleterious. Sustaining such a system whether it is found in cancer, parasitic disease or within the nervous system requires ATP in excess of the basal requirement. Sequestering L-lactate can disrupt the flux of ATP required to maintain the hyperactive state with the basal states fulfilling their energy needs through oxidation of some glucose or perhaps by more limited conversion of amino acids and fat to produce ATP. The benefit of PDLA or other agents that can sequester intermediates and can disrupt the non-equilibrium thermodynamic systems associated with hyperactivity could be very important.

### REFERENCES

1. Mehta M, Sonawat H M, Sharma S. Glycolysis in Plasmodium falciparum results in modulation of host enzyme activities. J Vector Borne Dis. September 2006; 43(3):95-103.
2. Slavic K, Krishna S, Derbyshire E T, Staines H M. Plasmodial sugar transporters as anti-malarial drug targets and comparisons with other protozoa. Malar J. 2011; 10:165.
3. Goldberg J S. Stereocomplexes Formed From Select Oligomers of Polymer d-lactic Acid (PDLA) and 1-lactate May Inhibit Growth of Cancer Cells and Help Diagnose Aggressive Cancers-Applications of the Warburg Effect. Perspect Medicin Chem. 2011; 5:1-10.
4. Egan T J. Haemozoin formation. Mol Biochem Parasitol. February 2008; 157(2):127-136.
5. Weissbuch I, Leiserowitz L. Interplay between malaria, crystalline hemozoin formation, and antimalarial drug action and design. Chem Rev. November 2008; 108(11):4899-4914.
6. Egan T J, Mavuso W W, Ncokazi K K. The mechanism of beta-hematin formation in acetate solution. Parallels between hemozoin formation and biomineralization processes. Biochemistry. January 9 2001; 40(1):204-213.
7. With permission from Dr. David M. Gooden
8. With permission from Dr. Noel S. Sturm
9. Slager J, Domb A J. Heterostereocomplexes prepared from d-poly(lactide) and leuprolide. I. Characterization. Biomacromolecules. September-October 2003; 4(5):1308-1315.
10. Slager J, Domb A J. Biopolymer stereocomplexes. Adv Drug Deliv Rev. Apr. 25, 2003; 55(4):549-583.
11. Tang F, Lane S, Korsak A, et al. Lactate-mediated glia-neuronal signalling in the mammalian brain. Nat Commun. 2014; 5:3284.
12. Jacob J N, Shashoua V E, Campbell A, Baldessarini R J. gamma-Aminobutyric acid esters. 2. Synthesis, brain uptake, and pharmacological properties of lipid esters of gamma-aminobutyric acid. J Med Chem. January 1985; 28(1):106-110.
13. Shashoua V E, Jacob J N, Ridge R, Campbell A, Baldessarini R J. Gamma-aminobutyric acid esters. 1. Synthesis, brain uptake, and pharmacological studies of aliphatic and steroid esters of gamma-aminobutyric acid. J Med Chem. May 1984; 27(5):659-664.
14. Ling B, Peng F, Alcorn J, Lohmann K, Bandy B, Zello G A. D-Lactate altered mitochondrial energy production in rat brain and heart but not liver. Nutr Metab (Lond). 2012; 9(1):6.

Having described my invention, I claim:

1. A method to sequester and form a stereocomplex when a single unit chiral molecule is in the vicinity of a polymer comprised of opposite chiral units.

2. The method of claim 1 where a single unit chiral molecule is in the vicinity of a polymer comprised of opposite enantiomeric chiral units.

3. The method of claim 1 where the polymer comprised of opposite chiral units is a drug that interrupts metabolic pathways.

4. The method of claim 1 where L-lactate is in the vicinity of poly D-lactic acid that disrupts glycolysis and inhibits the replication of a parasite.

5. A method that inhibits the sustenance of a hyperactive system found in disease states of the nervous system, such as but not limited to, mania, PTSD, depression and chronic pain by introducing a chiral polymer within the central nervous system.

6. The method of claim 7 in which the chiral polymer is polymer D-lactic acid.

7. The method of claim 7 in which the chiral polymer is a prodrug of polymer D-lactic acid.
